(19) United States
(12) Patent Application Publication
(10) Pub. No.: US 2004/0166087 A1
(43) Pub. Date: Aug. 26, 2004

(54) Asphalt composition with holding and masking agents

(76) Inventor: Henry Gembala, Boca Raton, FL (US)

Correspondence Address: Robert M. Downey, ROBERT M. DOWNEY, P.A., Suite 300, 601 South Federal Highway, Boca Raton, FL, 33432, US

(21) Appl. No.: 10/786,993

(22) Filed: Feb. 25, 2004

Related U.S. Application Data
(60) Provisional application No. 60/450,887, filed on Feb. 26, 2003.

(51) Int. Cl. A61L 9/00

(52) U.S. Cl. 424/76.1; 523/102

(57) ABSTRACT

Two specific types of ingredients are added to cold or hot melt asphalt or coal tar for the dual purposes of holding, reducing or complexing the obnoxious and toxic odors from asphalt while at the same time allowing a pleasant masking fragrance to predominate. The holding agents consist of various organic compounds which can bond or complex with and effectively hold onto other molecules. Typical complexing agents include dialkylgylcol alkyl ethers and dialkylphthalates. Typical fragrances include natural and synthetic oils or extracts such as lemon oil, orange oil, peppermint, spearmint, cinnamon, bubble gum and most other common fragrances. The concentrated extracts of fragrances consist of natural and synthetic materials having glycols, glycol ethers, alcohols, esters, aldehydes, ketones, and other compounds which can simply mask the obnoxious odors.

### BACKGROUND OF THE INVENTION

1. Field of the Invention

This invention relates generally to asphalt compositions and hot or cold process coal tar compositions and, more particularly, to a scented agent and a holding or complexing agent added to an asphalt composition or coal tar composition for reducing and masking objectionable odors.

2. Discussion of the Related Art

The need for odor reduction and masking in asphalt compositions and coal tar is well known in the construction industry. In particular, use of hot asphalt compositions and coal tar, either hot or cold process, during the installation or repair of roofing materials is known to generate obnoxious odors. In some instances, the unpleasant odors from use of asphalt compositions and coal tar becomes so overwhelming that it is necessary to evacuate a building, such as an office building or school, particularly when the obnoxious odors enter the air conditioning system. At present, there is no known effective means to eliminate the foul odors associated with asphalt compositions and coal tar during the installation of roofing materials.

### SUMMARY OF THE INVENTION

The present invention provides a very effective means to reduce and mask objectionable odors associated with asphalt compositions and coal tar compositions. More particularly, the present invention provides for the addition of an odor masking agent and a chemical complexing agent or holding agent to hot-mix asphalt compositions, cold lay asphalt, and coal tar, both hot and cold process so that the asphalt or coal tar composition will have a pre-dominant pleasant fragrance, as well as a chemical reduction of the unwanted obnoxious vapors. The scenting and complexing agents may be added to the compositions at the time of installation, such as when applying or repairing a roof structure. In one embodiment of the invention, the odor masking agent is a concentrated extract, such as vanilla extract or peppermint, as well as a holding or complexing agent such as diethyl phthalate or diethylene glycol methyl ether. A wide variety of similar esters, ethers, phthalates, alcohols, glycols, and ether organic compounds can be used for the purpose of hydrogen bonding or chemically complexing such odors. These compounds also complex the fragrances but not sufficiently to prevent the lower molecular weight fragrance components from being emitted. Thus the predominant odor will be due to the fragrance and not from the asphalt obnoxious odors.

### DETAILED DESCRIPTION OF THE PREFERRED EMBODIMENT

A scented agent and a holding or complexing agent are added to an asphalt mixture or coal tar, either hot or cold process, in order reduce and mask objectionable odors. The scented agent and the complexing agent may be added to the asphalt mixture or coal tar during the manufacturing process or, alternatively, as an additive at the time of installation, such as on a roof system. When used as an additive, the scented agent and complexing agent are mixed with the asphalt or coal tar, in a pot or vessel at the job site during the mixing process. The complexing agent chemically reacts or hydrogen bonds with the hydrocarbons, amines, sulfides, and other odor components present in asphalt and coal-tar. A mixture of the scented agent and holding agent was found to decrease unwanted obnoxious odors, while allowing the detection of the fragrance. The holding agent chemically bonds or complexes with the unwanted odors and, to a lesser extent, also holds onto the fragrance. However, the obnoxious odor is not only reduced, but also more effectively masked using both the scented agent and holding agent, as compared to use of only a fragrance.

In a preferred embodiment, the scented agent or the odor-masking agent consists of common fragrances, such as concentrated extracts of vanilla, peppermint, cinnamon, and synthetic bubble gum fragrance. The complexing or holding agents consist of relatively safe pure organic compounds such as diethyl phthalate, diethylene glycol methyl ether, and many other common esters, glycols, and alcohols which serve to chemically bond or complex with the obnoxious fumes. For example, a fifty-gallon batch of asphalt mixture or coal tar would be treated with one to two gallons of fragrance extract and one to two gallons of holding agent. When added in moderate amounts the fragrances and holding agents do not interfere with the performance or workability of the asphalt or coal tar products. However, the greater combustibility of the mixture makes it necessary to use more safeguards to avoid ignition during the melting process, especially with an open flame.

TABLE I
PROPOSED FORMULA “C” FOR ASPHALT
WITH MASKING AGENT AND HOLDING AGENT

| Component | Level (Wt. %) |
|---|---|
| Hot melt asphalt | 91.00% |
| Cinnamon oil | 4.5% |
| Diethyl phthalate | 4.5% |

Procedure:

Carefully melt asphalt and stir in the two other ingredients. Quickly mix well and cool. Formula can be remelted one time without losing significant fragrance.

TABLE II
PROPOSED FORMULA “D” FOR ASPHALT
WITH MASKING AGENT AND HOLDING AGENT

| Component | Level (Wt. %) |
|---|---|
| Hot melt asphalt | 91.00% |
| Bubble gum fragrance | 4.5% |
| Dipropylene Glycol Methyl Ether | 4.5% |

Procedure:

Carefully melt asphalt and stir in the two other ingredients. Quickly mix well and cool. Formula can be remelted one time without losing significant fragrance.

While the instant invention has been described in accordance with preferred and practical embodiments thereof, it is recognized that departures from the instant disclosure are contemplated within the spirit and scope of the present invention. In particular, a large number of scented agents and holding agents are contemplated for use in the present invention and are not limited to those described.

What is claimed is:

1. A composition for use in asphalt and coal tar mixtures comprising:
an odor masking agent; and
a holding agent structured to bond with obnoxious odors while allowing the odor masking agent to be at least partially released, wherein the odor masking agent and the holding agent combine to effectively reduce and mask objectionable odors of the asphalt and coal tar mixtures.

2. The composition as recited in claim 1 wherein said odor masking agent is selected from the group consisting of:
concentrated extract of vanilla; concentrated extract of peppermint; concentrated extract of cinnamon; and synthetic bubble gum fragrance.

3. The composition as recited in claim 1 wherein said odor masking agent is selected from the group consisting of:
natural and synthetic oils.

4. The composition as recited in claim 3 wherein said natural and synthetic oils include lemon oil and orange oil.

5. The composition as recited in claim 1 wherein said odor masking agent is selected from the group consisting of:
glycols, glycol ethers, alcohols, esters, aldehydes and ketones.

6. The composition as recited in claim 1 wherein said holding agent is selected from the group consisting of:
dialkylglycol alkyl ethers and dialkylphthalates.

7. The composition as recited in claim 1 wherein said holding agent is selected from the group consisting of:
pure organic compounds, including diethyl phthalate and diethylene glycol methyl ether.

8. A composition for adding to asphalt and coal tar mixtures comprising:
an odor masking agent; and
a holding agent structured to chemically bond with hydrocarbons, amines, sulfides, and other odor components present in the mixtures, wherein the odor masking agent and the holding agent combine to reduce and mask objectionable odors.

9. The composition as recited in claim 8 wherein said odor masking agent is selected from the group consisting of:
concentrated extract of vanilla;
concentrated extract of peppermint;
concentrated extract of cinnamon; and
synthetic bubble gum fragrance.

10. The composition as recited in claim 8 wherein said odor masking agent is selected from the group consisting of:
natural and synthetic oils.

11. The composition as recited in claim 10 wherein said natural and sythetic oils include lemon oil and orange oil.

12. The composition as recited in claim 8 wherein said odor masking agent is selected from the group consisting of:
glycols, glycol ethers, alcohols, esters, aldehydes and ketones.

13. The composition as recited in claim 8 wherein said holding agent is selected from the group consisting of:
dialkylglycol alkyl ethers and dialkylphthalates.

14. The composition as recited in claim 8 wherein said holding agent is selected from the group consisting of:
pure organic compounds, including diethyl phthalate diethylene glycol methyl ether.
